(19) United States
(12) Patent Application Publication
(10) Pub. No.: US 2021/0178056 A1
(43) Pub. Date: Jun. 17, 2021

(54) MEDICAL TUBING ORGANIZER

(71) Applicant: 3M INNOVATIVE PROPERTIES COMPANY, SAINT PAUL, MN (US)

(72) Inventors: SIMON S. FUNG, WOODBURY, MN (US); MITCHELL A. F. JOHNSON, MAPLEWOOD, MN (US); KRISTOPHER E. HUNTER, WOODBURY, MN (US)

(73) Assignee: 3M INNOVATIVE PROPERTIES COMPANY, SAINT PAUL, MN (US)

(21) Appl. No.: 17/067,850

(22) Filed: Oct. 12, 2020

Related U.S. Application Data
(60) Provisional application No. 62/946,675, filed on Dec. 11, 2019.

(51) Int. Cl. A61M 5/14; B32B 3/28; B32B 5/02; B32B 7/12; C09J 7/38; B32B 7/06

(52) U.S. Cl. CPC A61M 5/1418; B32B 3/28; B32B 5/02; B32B 7/12; B32B 2305/34; B32B 7/06; A61M 2209/084; B32B 2307/546; B32B 2535/00; C09J 7/38

(57) ABSTRACT

A tubing organizer is described herein. The tubing organizer comprising a cellular sheet comprising a base sheet having a first major surface and a second major surface, a top sheet periodically connected to the second major surface of the base sheet forming an array of fluid filled cells, wherein the fluid filled cells are separated by gutters, and an adhesive layer disposed on the first major surface of the base sheet.

### CROSS-REFERENCE TO RELATED APPLICATION

This application claims priority to U.S. Provisional Application Ser. No. 62/946,675, filed Dec. 11, 2019, the disclosure of which is incorporated by reference in its entirety herein.

### TECHNICAL FIELD

The present invention relates to medical organizer for tubing, cables and/or electrical leads, and more particularly to medical organizer retaining and organizing medical tubes for tubing, cables and/or electrical leads that can be adhered to a patient's skin.

### BACKGROUND

During hospitalization or other invasive medical treatments, such as chemotherapy, dialysis, etc., a patient maybe connected to one or more types of medical equipment by either tubing, cords and/or electrical leads. The tubing, cords, or electrical leads, which connects the patient to the equipment, such as oxygen, anesthesia, intravenous fluids, infusers, or monitoring equipment, can create a hazard for the patient and hospital personnel if allowed to dangle freely across the floor. Additionally, the tangle of tubing, cords, or electrical leads can make it difficult for a patient to move or change positions in their hospital bed and can pose similar problems when the clinicians need to move the patient for examination, transfer the patient for transportation to another location or to simply care for the patient. Medical lines, tubes, electrical leads or cables may be removed accidentally (e.g., dislodged) by tugging due to movement of the patient within the hospital bed, tripping or entanglement of a doctor, nurse, or visitor on the medical tubes, or by catching on devices, bedding or persons during transport of the patient. Therefore, it is best to organize the medical tubes, electrical leads or cables in an orderly manner through some securement devices.

Traditionally, tubing organizers comprise medium to high density molded plastic devices or in the form of two-dimensional dressings and medical tape such as film or fabric. However, the molded plastic devices lack flexibility which can be uncomfortable to the patient and may snag on hospital gowns or bedding which can result in pulling on attached cannulas, needles, etc., causing further discomfort and pain. More importantly, such rigid devices, when allowed to be pressed on elderly patients or patients who are clinically-compromised, can cause pressure injuries with a minimal amount of pressure. Such incidents can happen when patients inadvertently lie on such rigid organizers. The injuries can lead to a pressure ulcer event which is one of “never events” according to Centers for Medicare & Medicaid Services (CMS). The two-dimensional dressings and medical tape provide enhanced flexibility and have a lower overall profile, may not provide the sufficient constraint to the tubes, cords, etc. in the X-Y plane or on the patient's skin. From a classical constraint's standpoint, it is better to use a three-dimensional device to constrain a three-dimensional object.

### SUMMARY

A tubing organizer is described herein. The tubing organizer comprising a cellular sheet comprising a base sheet having a first major surface and a second major surface, a top sheet periodically connected to the second major surface of the base sheet forming an array of fluid filled cells, wherein the fluid filled cells are separated by gutters, and an adhesive layer disposed on the first major surface of the base sheet.

If used herein, the term “substantially” has the same meaning as “significantly,” and can be understood to modify the term that follows by at least about 75%, at least about 90%, at least about 95%, or at least about 98%. The term “not substantially” as used here has the same meaning as “not significantly,” and can be understood to have the inverse meaning of “substantially,” i.e., modifying the term that follows by not more than 25%, not more than 10%, not more than 5%, or not more than 2%.

Numeric values used herein include normal variations in measurements as expected by persons skilled in the art and should be understood to have the same meaning as “approximately” and to cover a typical margin of error, such as ±5% of the stated value.

Terms such as “a,” “an,” and “the” are not intended to refer to only a singular entity but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

### BRIEF DESCRIPTION OF DRAWINGS

FIG. 1 is a schematic cross-sectional view of a first embodiment of a tubing organizer according to an aspect of the present invention.

FIG. 2 is a schematic cross-sectional view of a second embodiment of a tubing organizer according to an aspect of the present invention.

FIGS. 3A-3B are schematic cross-sectional view of a third embodiment of a tubing organizer according to an aspect of the present invention.

FIG. 4 is a schematic cross-sectional view of a fourth embodiment of a tubing organizer according to the present invention.

FIG. 5 is a top view illustrating an exemplary application of an tubing organizer according to an aspect of the present invention.

FIG. 6 is a schematic cross-sectional view illustrating another exemplary application of a tubing organizer according to the present invention.

FIG. 7 is a schematic cross-sectional view illustrating yet another exemplary application of a tubing organizer according to the present invention.

FIGS. 8A-8B are two top views illustrating other exemplary applications of tubing organizers according to an aspect of the present invention.

FIGS. 9A-9D illustrate a method of using the exemplary tubing organizers of the present invention.

While the above-identified drawings and figures (which may or may not be drawn to scale) set forth embodiments of the invention, other embodiments are also contemplated, as noted in the discussion. In all cases, this disclosure presents the invention by way of representation and not limitation. It should be understood that numerous other modifications and embodiments can be devised by those skilled in the art, which fall within the scope of this invention.

### DESCRIPTION OF ILLUSTRATIVE EMBODIMENTS

Before any illustrative embodiments are described in detail, it is to be understood that the invention is not limited in its application to the details of construction and the arrangement of components set forth in the following description or illustrated in the following drawings. The invention is capable of other embodiments and of being practiced or of being carried out in various ways.

A tubing organizer includes a cellular sheet that is similar to the bubble wrap material used in packaging to capture and hold medical lines (i.e. medical tubing for air or oxygen supplies, surgical and ventricular drains and IV tubing and extensions, lines for negative pressure wound therapy which transport fluids and for vacuum, power cables and electrical leads and wires). In some aspects of the invention, the exemplary tubing organizer may also be used to hold medical devices that are attached to the medical lined held by the organizer. In an exemplary aspect, the tubing organizer comprises a skin safe adhesive disposed on the flat side of the cellular sheet so that the tubing organizer can be adhered to the patient's skin.

FIG. 1 shows a first embodiment of an exemplary tubing organizer 100 of the present invention. Tubing organizer 100 comprises a cellular sheet 110 composed of two plastic sheets, i.e. base sheet 112 and top sheet 115. The base sheet has a first major surface 112a and a second major surface 112b wherein the top sheet is periodically connected to the second major surface of the base sheet providing an array of fluid filled cells, to be formed therebetween. An adhesive layer 130 is disposed on the first major surface of base sheet 112. The pockets 117 can be formed by periodically heat sealing the top sheet to the bottom sheet around the perimeter of each pocket to form gutters 118 and filling each pocket to a desired fill ratio with a fluid such as air, nitrogen, water or saline. In some embodiments, the fill ratio can be 75-95% of the pockets full capacity to provide cells having the necessary compliance for the routing of medical lines in the tubing organizer. In an exemplary aspect, base sheet 112 can be substantially planar with the fluid filled pockets being formed predominately in the top sheet of cellular sheet 110, such that the fluid filled cells protrude from the surface of base sheet 112 by the height, h, of the cells.

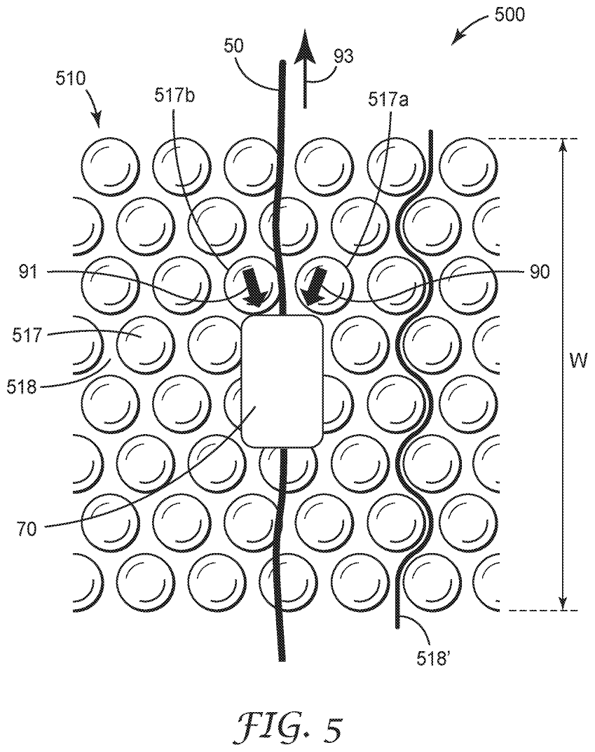

In an exemplary embodiment, fluid filled cells 117 can be distributed across base sheet 112 in a two-dimensional array with a network of crossing gutters 118 running between adjacent cells. The fluid filled cells may be aligned in parallel rows and columns such that the gutters form a continuous linear path across the width of the exemplary tubing organizer. In other embodiments, the cells can be arranged in staggered rows creating gutters having a more tortuous path across the width, W, of the organizer, such as is shown in FIG. 5.

In still other embodiments, the fluid filled cells can be distributed across the tubing organizer in a one-dimensional array. In these embodiments the fluid filled cells may have an elongated structure creating gutters that extend across the width of the tubing organizer. These organizers are useful in situations where the only concern is the management of medical lines such as medical tubing, cords or electrical leads.

In an exemplary aspect, tubing organizer 100 is flexible so that it can conform to curved or irregular surface. The exemplary tubing organizer can flex and conform to the patient's skin even when the patient shifts or moves in their bed, while being examined or transported to other locations in the hospital.

The fluid filled cells or pockets 117 can be characterized by their height and their cross-section parallel to the base sheet. The cell's cross-section can be generally circular, elliptical, rectangular, or other polygonal shape selected to hold the medical lines (medical tubing, cords, or electrical leads) or devices (infusers, catheters, line splitters, and the like).

Adhesive layer 130 can comprise a first pressure sensitive layer which skin safe (i.e. the adhesive is non-irritating and can be cleanly removed from the skin surface without damaging the skin's surface). In some embodiments, the adhesive layer is moisture permeable. In an exemplary aspect, adhesive layer 130 may be patterned or perforated to allow moisture to pass therethrough or wick moisture away from the area under the tubing organizer. For example, when the adhesive is patterned, channel can be formed in the adhesive to wick moisture away from the area under the tubing organizer. An optional release liner 150 can be disposed over adhesive layer 130 to prevent contamination of the adhesive layer prior to adhering the exemplary tubing organized onto the patient.

Tubing organizer 100 can further comprise a cover sheet 120 that can be adhered to the tops of the fluid filled cells 117 to secure medical tubing 50, 50′ within the gutters 118 between adjacent fluid filled cells. The cover sheet can comprise pressure sensitive adhesive layer 122 comprising a second adhesive disposed on a backing 124. For example, the adhesive coated top sheet comprises an elastomeric film coated with a pressure sensitive adhesive, wherein the elastomeric film is selected from one of a polyurethane film, polyethylene or other thermoplastic films. In some embodiments, the cover sheet can be a separate material that is adhered over the cellular sheet after the medical lines, and/or devices are positioned on the cellular sheet. In other embodiments, a portion of the cover sheet can be pre-attached to tubing organizer with the remaining portion being adhered to the cellular sheet after the medical lines, and/or devices are positioned on the cellular sheet. When cover sheet is pre-attached to tubing organizer, the cover sheet can include a release liner disposed on the unattached adhesive layer of said cover sheet.

In some embodiments, the second adhesive layer can be a pressure sensitive adhesive comprising either a repositionable adhesive or a quasi-permanent adhesive. A repositionable adhesive allows the cover sheet to be peeled back from the cellular sheet to allow inspection and/or repositioning of the line disposed in the exemplary organizer or can allow the addition of new medical lines to the organizer. Exemplary repositionable adhesives which can be used as the second adhesive on coversheet can be polyurethane adhesive, acrylic adhesives, silicone adhesive, or the like having a peel strength that is less than the cohesive strength of the cellular sheet.

A quasi-permanent adhesive builds strength over time to the point where any attempt to pull back the cover sheet would result removal or destruction of the cellular sheet. Use of a quasi-permanent adhesive could be useful to prevent patients from disturbing the medical lines being held by the exemplary tubing organizer. Since adhesion builds over time, the quasi-permanent adhesive can allow clinicians time initially to peel the cover layer back to reposition lines, but after a set time has passed the peel strength of the adhesive will increase to a value that is greater than the cohesive strength of the cellular sheet. Exemplary quasi-permanent adhesives which can be used as the second adhesive on coversheet can be acrylic adhesives, synthetic rubber adhesives or the like that have a peel strength that is greater than the cohesive strength of the cellular sheet. In some embodiments, the synthetic rubber is at least one of polyisoprene rubber, polybutadiene rubber, styrene-isoprene rubber, styrene-butadiene rubber (SBR), polychloroprene rubber, nitrile rubber, and mixtures thereof, such as is described in International Application No. PCT/US2019/045521, herein incorporated by reference in its entirety.

Alternatively, a quasi-permanent adhesive may be a permanent adhesive that is combined with a means of controlling the permanence such as softening the adhesive with heat to facilitate removal (with or without destruction of the cell or coversheet).

In some exemplary embodiments, the medical tubing 50 may be larger than the width, w, of gutters 118 such that the sidewalls of the adjacent cells on either side of the tubing are deformed which can help to lock tubing 50 within the gutter. In other aspects, medical tubing 50′ can be smaller than the width of the gutter such that the cover sheet is responsible for keeping the tubing within the gutter.

FIG. 2 shows exemplary tubing organizer 200 of the present invention. Tubing organizer 200 comprises a cellular sheet 210 composed of two plastic sheets, i.e. a generally planar base sheet 212 and top sheet 215, having an array of individual fluid filled cells or pockets 217 formed therebetween and a skin safe adhesive layer 230 disposed on the exposed surface of base sheet 212. The pockets 217 are formed by heat sealing the top sheet to the bottom sheet around the perimeter of each pocket to form gutters 218 and filling each pocket to a desired fill ratio with a fluid such as air, nitrogen, water or saline. The fluid filled cells or pockets 217 can be characterized by their height, h, and their cross-section parallel to the base sheet as well as the spacing between adjacent cells (i.e. the width, w, of the gutters 218). An optional release liner 250 can be disposed over adhesive layer 230 to prevent contamination of the adhesive layer prior to adhering the exemplary tubing organized onto the patient.

Tubing organizer 200 further comprises an adhesive disposed in the bottoms of the gutters which will be referred to herein as gutter adhesive 219. Gutter adhesive 219 can be dispensed into the gutters during the manufacture of tubing organizer 200 in the form of a hotmelt adhesive, a solvent based adhesive, or a water-based adhesive and either cooled or dried to its final form. Gutter adhesive can comprise the second adhesive described above. The gutter adhesive can be a layer can be a pressure sensitive adhesive comprising either a repositionable adhesive or a quasi-permanent adhesive.

When installing the medical lines 50, 50′ into the gutters, a downward pressure, P, is applied to the medical lines to ensure good contact between the medical lines and gutter adhesive 219 to secure the medical lines in the tubing organizer.

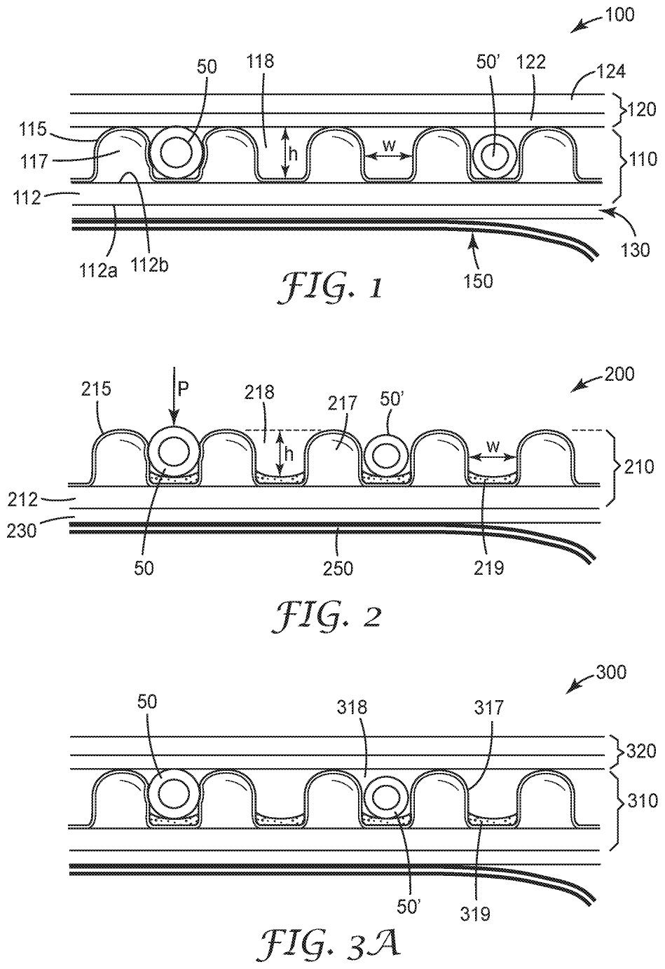

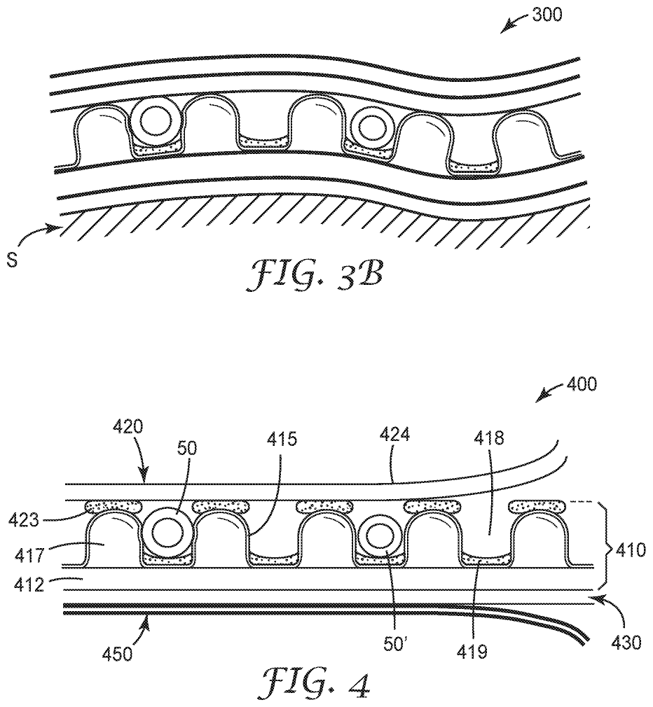

FIGS. 3A and 3B show exemplary tubing organizer 300 of the present invention. Tubing organizer 300 is similar to tubing organizer 200 shown in FIG. 2 with the exception that tubing organizer 300 comprises a cover sheet 320 that can be adhered to the tops of the fluid filled cells 317 of cellular sheet 310 covering medical lines 50, 50′ which are disposed within gutters 318 and held in place by gutter adhesive 319. FIG. 3B illustrates the conformability of tubing organizer 300 when it is attached to a patient's skin, S.

FIG. 4 shows another exemplary tubing organizer 400 that comprises a cellular sheet 410 formed from a base sheet 412 and a top sheet 415 and having an array of individual fluid filled cells 417 formed therebetween. A skin safe adhesive layer 430 is disposed on the exposed surface of base sheet 412. An optional release liner 450 can be disposed over adhesive layer 430 to prevent contamination of the adhesive layer prior to adhering the exemplary tubing organized onto the patient. Tubing organizer 400 further comprises gutter adhesive 419 disposed in the bottoms of the gutters 418 between adjacent cells.

Tubing organizer 400 can further comprises adhesive caps 423 disposed on top of each of the fluid filled cells 417. The adhesive caps may be formed by laminating a patterned adhesive sheet on top of the cellular sheet and removing the portion that is not adhered to the tops of the fluid filled cells. In an alternative aspect, the adhesive caps and the gutter adhesive can be introduced simultaneously by a conventional spray coating process. When the adhesive caps and the gutter adhesive are applied simultaneously, the adhesive caps and the gutter adhesive will comprise the same adhesive. However, it is also contemplated that the adhesive caps and the gutter adhesive can be different adhesives applied at different times in the manufacture of the exemplary tubing organizer.

Cover sheet 420 that can be adhered to the tops of the fluid filled cells 417 by adhesive caps 423 to cover medical tubing 50, 50′ within the gutters between adjacent fluid filled cells. In this embodiment cover sheet 420 comprises a polymeric cover film with or without an adhesive layer. In an alternative embodiment, the adhesive caps can be a contact adhesive which can combined with a cover sheet that comprises a backing layer and a contact adhesive layer (not shown).

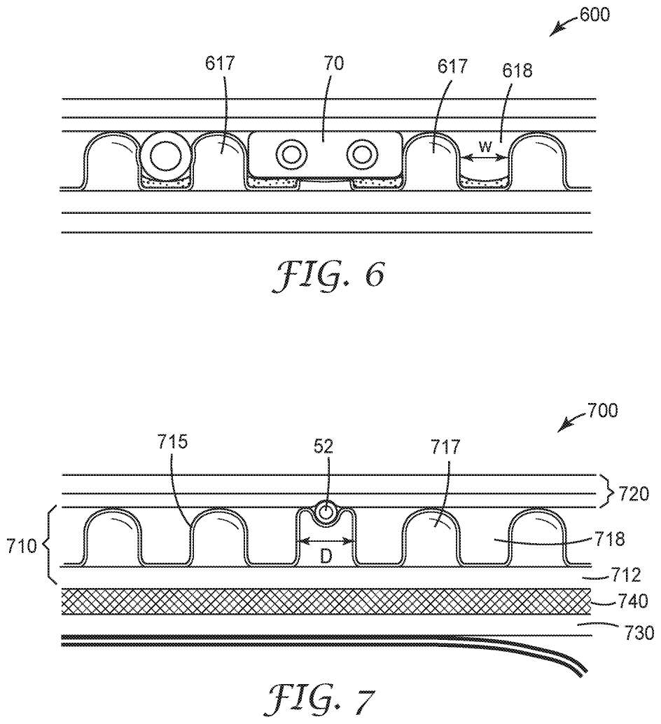

FIG. 5 is a top view of tubing organizer 500. Tubing organizer 500 comprises a cellular sheet 510 made up of a two-dimensional array of fluid filled cells 517 that are separated by gutters 518. In particular, fluid filled cells 517 are arranged in staggered rows creating gutters having a tortuous path 518′ across the width, W, of tubing organizer 500. FIG. 5 shows how the exemplary tubing organizers can be used to hold a medical device such as a bushing/valve, line splitter, Luer lock connection device, catheters such as including Foley, vascular, arterial and others common catheters, cannula, etc. The medical device can be positioned against one or more fluid filled cells, such as fluid filled cells 517a, 517b. When the device 70 is larger than the width, w, of a gutter 618, the clinician may pop or deflate one or more of the fluid filled cells as is illustrated in FIG. 6 so that the device fits between a plurality of the remaining fluid filled cells 617. The fluid filled cells provide an in-plane counteracting force (indicated by arrows 90, 91) when medical line 50 is pulled away from a skin insertion site (indicated by arrow 93) as shown in FIG. 5.

In contrast, conventional taping methods used for holding medical devices such as catheters do not provide this same in-plane counteracting force. For example, adhesive tape typically holds devices via the shear characteristics of the adhesive disposed on top of the device which is out of plane to the applied force which can limit the adhesive tape's capability to properly restrain the device. In other aspects, other conventional organizer devices, which are designed to provide a similar type of mechanical in-plane counteracting force, are rigid in nature may cause pressure related injuries as described earlier.

FIG. 7 shows another exemplary tubing organizer 700 that comprises a cellular sheet 710 formed from a base sheet 712 and a top sheet 715 and having an array of individual fluid filled cells 717 formed therebetween. FIG. 7 shows how tubing organizer 700 can be used to hold small medical lines such as electrical lead 52 which are smaller than the fluid filled cells. For example, when the fluid filled cells have a circular cross section and the electrical lead 52 is smaller than the diameter of the fluid filled cells, the electrical lead may be run along the tops of the fluid filled cell and held in place by cover sheet 720.

In some embodiments, the exemplary tubing organizers of the present disclosure may further comprise a porous substrate 740 disposed between the adhesive layer 730 and the base sheet 712 of the cellular sheet 710. In an exemplary aspect, the porous substrate can be an open cell foam, a nonwoven fabric, a woven fabric or a knitted fabric. In some aspects, an additional adhesive layer (not shown) can be used to secure the porous substrate to the base layer. In other aspects, the porous substrate may be directly laminated to the base layer (e.g. thermally or chemically welded).

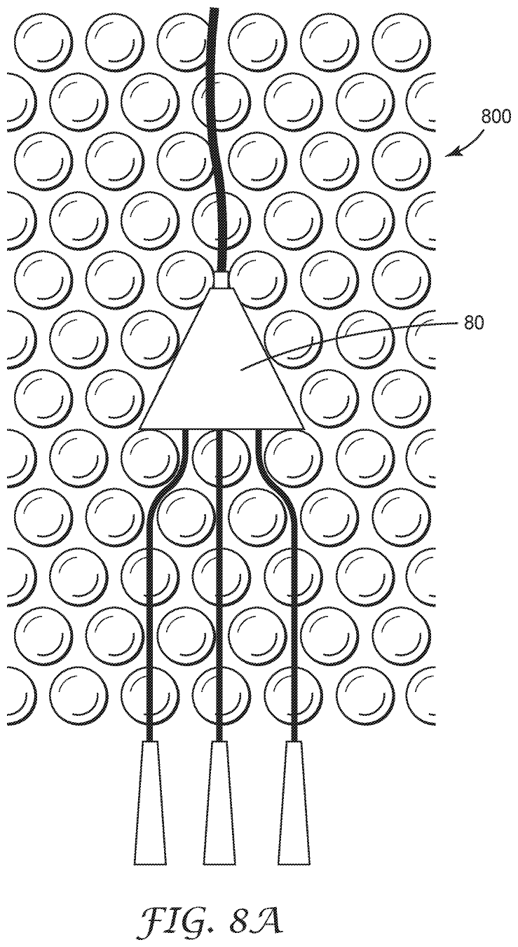

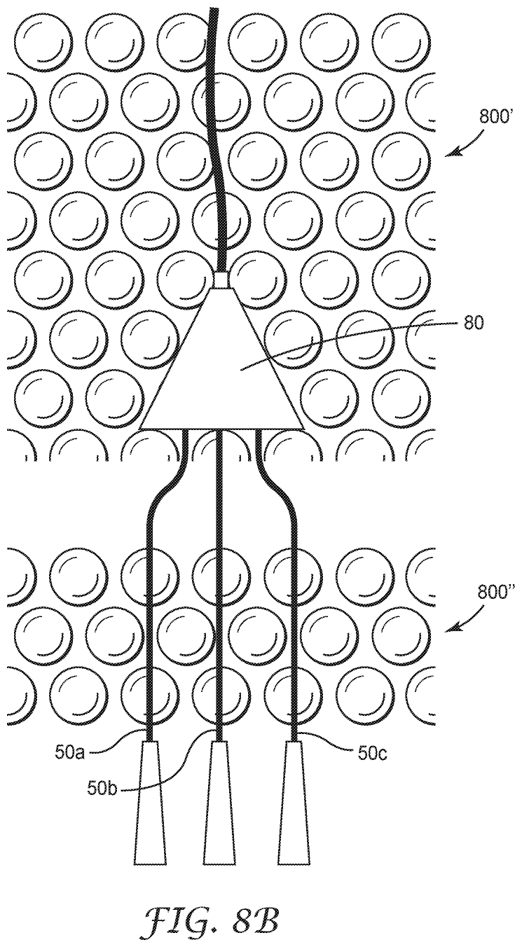

FIGS. 8A-8B are views illustrating other exemplary applications of tubing organizers described above. FIG. 8A shows a three-way line splitter 80 being held by a single tubing organizer 800, while FIG. 8B shows using two tubing organizers 800′, 800″ to hold the same device. Tubing organizer 800′ is being used to secure the three-way line splitter 80 and tubing organizer 800″ is used to manage the individual medical lines 50a-50c. The latter arrangement may be advantageous when the medical lines 50a-50c are long or when the device is being put near one of the patient's joints where it might be advantageous to have that extra degree of flexibility.

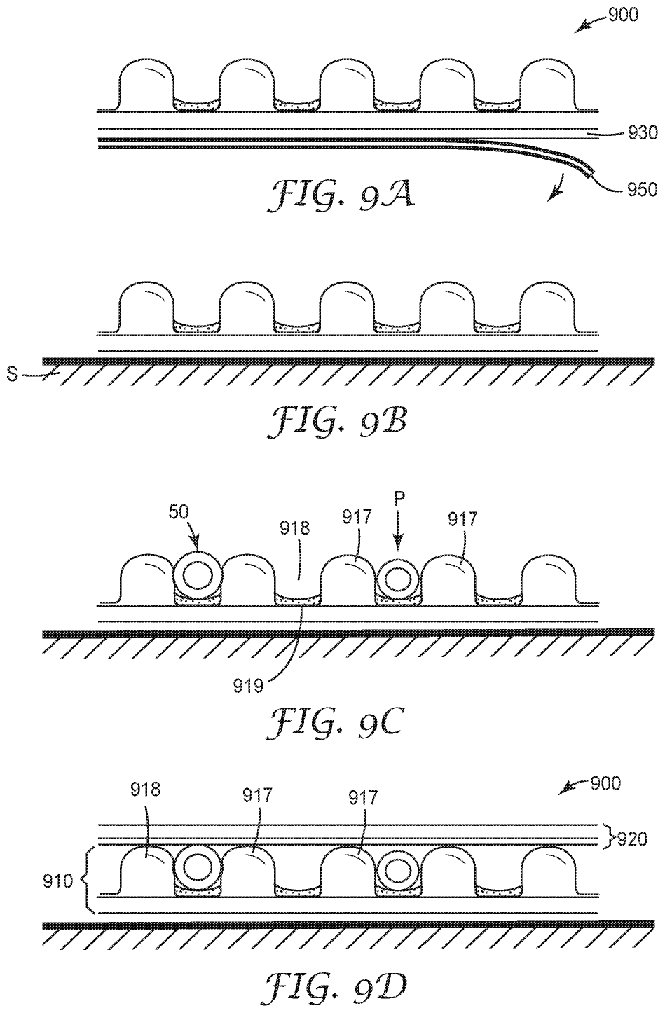

FIGS. 9A-9D illustrate an exemplary method of using a tubing organizer according to the present invention. First, release liner 950 is peeled off of the skin-safe adhesive layer 930 of tubing organizer 900 (FIG. 9A). The tubing organizer is applied to the patient's skin, S, (FIG. 9B). Next the medical lines 50 are placed in the gutters 918. When the tubing organizer includes a gutter adhesive 919, a small amount of downward pressure, P, is applied to the medical lines to ensure good contact between the medical lines and gutter adhesive to secure the medical lines in the tubing organizer, as illustrated in FIG. 9C. Finally, an optional cover sheet 920 may be applied to the tops of the fluid filled cells 917 to cover the gutters 918, medical lines 50 and or medical devices (not shown) and to complete installation of tubing organizer 900 (FIG. 9D).

The exemplary tubing organizers, described herein, can allow clinicians to secure medical lines and devices in a convenient and adjustable way while being able to see the lines/devices and skin underneath the tubing organizer.

### Cellular Sheet

In one or more embodiments as described herein, the plastic sheets of the cellular sheet should be suitable for prolonged contact with the skin and should be sterilizable, such as by the application of heat, chemicals or radiation. In a preferred embodiment, the first and second film layers are formed of a heat-sealable thermoplastic material selected from the group consisting of polyethylene, polyvinyl chloride, polypropylene, nylon, cellulose acetate, polystyrene, and any copolymers and materials having similar heat-sealable characteristics.

In some embodiments, the first and second film layers can be coated with a thin layer material to make the first and second film layers more impermeable to the fluid disposed in the fluid filled cells. For example, when the fluid in the fluid filled cells is air, the coating can be formed of a gas-impervious material such as polyvinylidene chloride and vinylidene chloride copolymers.

### Cover Sheet

Suitable cover sheet materials can include polymer films or can be adhesive coated materials such as medical tapes or medical dressings. In an exemplary aspect, the cover sheet should be flexible enough to conform to the surface of the cellular sheet and any medical lines and/or devices being secured by the exemplary organizer. The cover sheet can be sufficiently translucent or transparent to allow viewing of the medical lines and/or devices being secured by the exemplary organizer without having to remove the cover sheet. In some embodiments, the cover sheet comprises a writeable surface that allows clinicians to identify the medical lines held by the tubing organizer as well as potentially allowing identification of when and by whom the organizer was placed on the patient's skin.

Exemplary polymer films usable for the cover sheet include polyethylene, polyurethane, polypropylene, nylon, polyester and copolymers having similar transparent, conformability, flexibility or other similar properties.

Exemplary medical tapes that can be used as the cover film include rayon-based, cellulose acetate-based, polyethylene-based, and copolymers having similar properties. Exemplary examples of suitable medical tapes include 3M™ Transpore™ Surgical Tapes, 3M™ Micropore™ Surgical Tapes, and 3M Durapore™ Medical Tapes; all of which are available from 3M Company (St. Paul, Minn.).

In some embodiments, adhesive coated cover sheet can comprise an elastomeric film coated with a pressure sensitive adhesive, wherein the elastomeric film is selected from one of a polyurethane film, polyester, nylon and copolymers having similar properties. An exemplary adhesive coated cover sheet can comprise a 3M™ Tegaderm™ Transparent Film Dressing available from 3M Company (St. Paul, Minn.).

### Skin-Facing Adhesive

The adhesive layer used to attach the exemplary tube securing device to the patient's skin can be referred to as a skin facing adhesive. Suitable skin facing adhesives include any adhesive (or combination of adhesives) that provides acceptable adhesion to skin and is acceptable for use on skin (e.g., the adhesive should preferably be non-irritating and non-sensitizing). Suitable adhesives are pressure sensitive and in certain embodiments have a relatively high moisture vapor transmission rate to allow for moisture evaporation. Suitable pressure sensitive adhesives include those based on acrylates, urethane, hydrogels, hydrocolloids, block copolymers, silicones, rubber based adhesives (including natural rubber, polyisoprene, polyisobutylene, butyl rubber etc.) as well as combinations of these adhesives. The skin-facing adhesive may contain tackifiers, plasticizers, rheology modifiers as well as active components including for example an antimicrobial agent.

The pressure sensitive adhesives that may be applied to the skin can comprise the acrylate copolymers described in U.S. Pat. No. RE 24,906, particularly a 97:3 isooctyl acrylate:acrylamide copolymer. Another example may include a 70:15:15 isooctyl acrylate:ethyleneoxide acrylate:acrylic acid terpolymer, as described in U.S. Pat. No. 4,737,410 (Example 31). Other potentially useful adhesives are described in U.S. Pat. Nos. 3,389,827; 4,112,213; 4,310,509; and 4,323,557. Inclusion of medicaments or antimicrobial agents in the adhesive is also contemplated, as described in U.S. Pat. Nos. 4,310,509 and 4,323,557.

Silicone adhesive can also be used. Generally, silicone adhesives can provide suitable adhesion to skin while gently removing from skin. Suitable silicone adhesives are disclosed in U.S. Pat. No. 9,359,529 (Liu et al.); U.S. Pat. No. 8,822,560 (Seth et al.); U.S. Pat. No. 8,822,559 (Zoller et al.), U.S. Pat. No. 7,407,709 (Zhou et al.), and US Patent Publication US 2011/0206924 (Liu et al.).

The pressure sensitive adhesives may, in some embodiments, transmit moisture vapor at a rate greater than or equal to that of human skin. While such a characteristic can be achieved through the selection of an appropriate adhesive, it is also contemplated that other methods of achieving a high relative rate of moisture vapor transmission may be used, such as pattern coating the adhesive on the porous substrate (as described in. for example, U.S. Pat. No. 4,595,001). Other potentially suitable pressure sensitive adhesives may include blown-micro-fiber (BMF) adhesives such as, for example, those described in U.S. Pat. No. 6,994,904. The pressure sensitive adhesive used in contact with skin may also include one or more areas in which the adhesive itself includes structures such as, for example, the microreplicated structures described in U.S. Pat. No. 6,893,655.

While adhesives and adhesive articles have shown themselves to be very useful for medical applications, there are also issues in the use of adhesives and adhesive articles. Medical adhesive-related skin injury (MARSI) can have a negative impact on patient safety. Skin injury related to medical adhesive usage is a prevalent but under recognized complication that occurs across all care settings and among all age groups. In addition, treating skin damage is costly in terms of service provision, time, and additional treatments and supplies.

Medical adhesive articles such as tapes, dressings, etc. can be simply defined as a pressure-sensitive adhesive and a backing that acts as a carrier for the adhesive. The US Food and Drug Administration more specifically defines a medical adhesive tape or adhesive bandage as “a device intended for medical purposes that consists of a strip of fabric material or plastic, coated on one side with an adhesive, and may include a pad of surgical dressing without a disinfectant. The device is used to cover and protect wounds, to hold together the skin edges of a wound, to support an injured part of the body, or to secure objects to the skin.”

Skin injury occurs when the superficial layers of the skin are removed along with the medical adhesive product, which not only affects skin integrity but can cause pain and the risk of infection, increase wound size, and delay healing, all of which reduce patients' quality of life. While the pathophysiology of MARSI is only partially understood, skin injury results when the skin to adhesive attachment is stronger than skin cell to skin cell attachment. When adhesive strength exceeds the strength of skin cell to skin cell interactions, cohesive failure occurs within the skin cell layer.

The intrinsic characteristics of all components of an adhesive product should be considered to address the factors that may lead to MARSI. Properties of the adhesive to be considered include cohesiveness over time and the corresponding adhesion strength; properties of the tape/backing/dressing to be considered include breathability, stretch, conformability, flexibility, and strength.

### Porous Substrate

The porous substrate can be an open cell foam, a high moisture vapor permeable film, woven, knitted, or nonwoven fabric. For example, U.S. Pat. No. 5,088,483 discloses a permanent adhesive as a reinforcement that could be used as the porous substrate.

One example of nonwoven useful as porous substrate 740 is a high strength nonwoven fabric available from E. I. Dupont de Nemours & Company of Wilmington, Del. under the trademark Sontara, including Sontara® 8010, a hydroentangled polyester fabric. Other suitable nonwoven webs include a hydroentangled polyester fabric available from Veratec, a division of International Paper of Walpole, Mass. Another suitable nonwoven web is the nonwoven elastomeric web described in U.S. Pat. No. 5,230,701, herein incorporated by reference in its entirety.

One example of a high moisture vapor permeable film is described in U.S. Pat. Nos. 3,645,835 and 4,595,001, the disclosures of which are herein incorporated by reference. In general, a high moisture vapor permeable film can have a high permeability to water vapor, while remaining generally impermeable to liquid water so that microbes and other contaminants are sealed out from the area under the porous substrate/tubing holder. In one or more embodiments, the high moisture vapor permeable film can be an elastomeric polyurethane, polyester, or polyether block amide films. These films combine the desirable properties of resiliency, elasticity, high moisture vapor permeability, and transparency. A description of these high moisture vapor permeable films can be found in U.S. Pat. Nos. 5,088,483 and 5,160,315, the disclosures of which are hereby incorporated by reference.

In one or more embodiments as described herein, one or more pressure-sensitive adhesives may be provided on one or both major surfaces of the porous substrate to form a high moisture vapor permeable film/adhesive composite. Such composites may preferably transmit moisture vapor at a rate equal to or greater than human skin such as, for example, at a rate of at least 300 g/m²/24 hrs. at 37° C./100-10% relative humidity (RH), or at least 700 g/m²/24 hrs. at 37° C./100-10% RH, or at least 2000 g/m²/24 hrs. at 37° C./100-10% RH using the inverted cup method as described in U.S. Pat. No. 4,595,001. Perforated films or pattern coated adhesives may be used to increase the moisture vapor transmission of the backing layer and/or film/adhesive composite.

In one or more embodiments, the porous substrate may be kept relatively thin to, e.g., improve conformability. For example, when the backing layer is formed of high moisture vapor permeable polymeric film, the thickness of the porous substrate can be 200 micrometers or less, or 100 micrometers or less, potentially 50 micrometers or less, or even 25 micrometers or less.

### Optional Components

A variety of optional components may be included in one or more embodiments of tubing holders as described herein. For example, release liners may be included that covers all or a portion of any exposed adhesives to prevent contamination of those adhesives. Suitable release liners can be made of Kraft papers, polyethylene, polypropylene, polyester or composites of any of these materials. In one embodiment, the liners are coated with release agents such as fluorochemicals or silicones. For example, U.S. Pat. No. 4,472,480, the disclosure of which is hereby incorporated by reference, describes low surface energy perfluorochemical liners. In one embodiment, the liners are papers, polyolefin films, or polyester films coated with silicone release materials.

All references and publications cited herein are expressly incorporated herein by reference in their entirety into this disclosure, except to the extent they may directly contradict this disclosure. Although specific illustrative embodiments have been described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims.

What is claimed is:

1. A tubing organizer comprising:
a cellular sheet comprising a base sheet having a first major surface and a second major surface;
a top sheet periodically connected to the second major surface of the base sheet forming an array of fluid filled cells, wherein the fluid filled cells are separated by gutters; and
an adhesive layer disposed on the first major surface of the base sheet.

2. The tubing organizer according to claim 1, wherein the base sheet is substantially flat and the fluid filled cells protrude from a second major surface of the base sheet.

3. The tubing organizer according to claim 1, wherein the adhesive layer comprises a first pressure sensitive adhesive.

4. The tubing organizer according to claim 1, wherein adhesive layer is patterned to facilitate dispersing of moisture vapor.

5. The tubing organizer according to claim 1, further comprising a porous substrate disposed between the adhesive layer and the base sheet.

6. The tubing organizer according to claim 5, wherein the porous substrate is one of an open cell foam, a nonwoven fabric, a woven fabric or a knitted fabric.

7. The tubing organizer according to claim 5, further comprising a second adhesive layer attaching the porous layer to the base sheet.

8. The tubing organizer according to claim 1, wherein the fluid filled cells are filled with air, nitrogen, water, or saline.

9. The tubing organizer according to claim 1, wherein the tubing organizer is flexible and can conform to curved or irregular surfaces.

10. The tubing organizer according to claim 1, further comprising a cover sheet adhered on top of the fluid filled cells.

11. The tubing organizer according to claim 10, wherein the cover sheet is a polymeric cover film.

12. The tubing organizer according to claim 10, wherein the cover sheet comprises an elastomeric film coated with a pressure sensitive adhesive.

13. The tubing organizer according to claim 1, further comprising a release liner disposed on the adhesive layer.

14. The tubing organizer according to claim 1, further comprising a gutter adhesive disposed in the gutters of the cellular sheet.

15. The tubing organizer according to claim 14, wherein the gutter adhesive comprises a second pressure sensitive adhesive.

16. The tubing organizer according to claim 14, wherein the gutter adhesive is characterized by a peel strength that is greater than the cohesive strength of the cellular sheet.

17. A method of using the tubing organizer according to claim 1, the method comprising:
adhering the tubing organizer to a patient's skin; and
placing at least one medical line or medical device in the cellular sheet.

18. A method of claim 17, further comprising applying pressure to the medical line or device to promote adhesion to a gutter adhesive disposed within gutters of the cellular sheet.

19. A method of claim 17, further comprising breaking one or more fluid filled cells to allow placement of medical lines or devices having dimensions greater than the width of the gutters.

20. A method of claim 19, further comprising adhering a cover sheet to tops of the fluid filled cells to cover the gutters.
